(19) United States
(12) Patent Application Publication
(10) Pub. No.: US 2009/0214633 A1
(43) Pub. Date: Aug. 27, 2009

(54) NANOPARTICLES WITH LIPID CORE AND POLYMER SHELL STRUCTURES FOR PROTEIN DRUG DELIVERY PREPARED BY NANOENCAPSULATION

(75) Inventors: Soon-Hong Yuk, Daejeon (KR); Keun-Sang Oh, Daejeon (KR)

Correspondence Address: NIXON PEABODY, LLP, 401 9TH STREET, NW, SUITE 900, WASHINGTON, DC, 20004-2128, US

(73) Assignee: Hunnam University Institute for Industry Academia Cooperation, Daejeon (KR)

(21) Appl. No.: 12/296,100

(22) PCT Filed: Dec. 1, 2006
(86) PCT No.: PCT/KR2006/005150

§ 371 Date: Apr. 2, 2009

(30) Foreign Application Priority Data
Apr. 5, 2006 (KR) 10-2006-0030944

(51) Int. Cl. A61K 9/127; A61K 38/16

(52) U.S. Cl. 424/450; 514/12; 977/773

(57) ABSTRACT

The present invention relates to an application of stabilized nanoparticles with a lipid core and shell structures as protein drug carriers, wherein the nanoparticles are prepared by producing nano-sized particles from lecithin obtained from natural soybean, and then adsorbing polaxamer thereon. Here, lecithin is used as an ingredient of core structure, polaxamer is used as an ingredient of shell structure, and both ingredients are applicable to a human body. Thus-obtained nanoparticles having a lipid core and shell structures are applicable in the clinical application. Moreover, said nanoparticles are suitable for use as a drug carrier or a diagnostic agent because they are produced in an aqueous solution without organic solvents.

### TECHNICAL FIELD

The present invention relates a method for preparing nanoparticles suitable for protein drug delivery from a biocompatible macromolecule and a biodegradable natural material by using a molecular assembly technique. More specifically, the present invention relates to a method for preparing nano-sized particles by blending a lecithin extracted from soybeans with a protein drug, and a method for preparing nanoparticles with a core and shell structures for protein drug delivery by using a molecular assembly technique in order to improve the stability of a protein drug and adjust the drug release properties thereof.

### BACKGROUND ART

Thanks to advances in biotechnology and molecular biology, the preparation of recombinant DNA was made possible, and thereby new protein drugs using the DNA are being developed. Unlike other synthetic drugs, a protein drug can exert its efficacy only when its three-dimensional structure is maintained in an aqueous solution. However, it is difficult to store a protein drug in a way such that its three-dimensional structure can be maintained. In addition, due to the above characteristic, a protein drug has a limitation in use that it can be delivered into the human body only by injection. As a result, despite its excellent performance, there have been many obstacles in using a protein drug. Moreover, because many protein drugs have a decreased stability after they are administered to the human body, there is a limitation that multiple injections are required.

In order to overcome the above problems, many studies have been done on the extended-release or sustained-release of drugs, which maintain efficacy by slowly releasing the drug in the human body. Also, the extended-release or sustained-release drug delivery system, wherein a drug is loaded into nanoparticles, prepared from biodegradable or biocompatible polymers, is being praised.

For the past several years, a biodegradable polymer, poly(d,l-lactide-co-glycolide) (PLGA), has been used in a pharmaceutical industry as a material to deliver biologicals, such as a water-soluble or water-insoluble polymer drug, a vaccine serum for prevention, diagnosis and treatment use, etc. into the human body in a particulate form. (U.S. Pat. No. 5,876,761, U.S. Pat. No. 6,201,065, U.S. Pat. No. 6,270,795, U.S. Pat. No. 6,238,702, and U.S. Pat. No. 6,248,345) Recently, the US FDA has approved a PLGA microsphere for 30-day delivery system of leuprolide acetate (Lupran Depot (registered trade mark)) to treat a prostate cancer. A useful review on the potential of polymer microencapsulation technology for vaccine use is found in Vaccine, 1994, volume 12, number 1, pages 5-11, by William Morris et al.

However, because of hydrophobic nature of poly(d,l-lactide-co-glycolide) (PLGA), PLGA is not proper as a candidate material for protein-drug carrier, which requires hydrophilic conditions. In addition, it has been reported that the protein drug loaded in said polymer nanoparticles exhibits a decreased activity. For the past ten years, the studies for the development of microparticles/nanoparticles have mainly focused on how to load a protein drug into microparticles/nanoparticles without the decrease of its activity, but even the recently-registered patents, U.S. Pat. No. 6,586,011 and U.S. Pat. No. 6,616,944 do not solve the problem of the decrease of the activity. That is, it can be known that in the field of developing polymers to minimize the decrase of the protein activity, a plenty of time and efforts are required to obtain the stability of the protein drug loaded thereinto.

Meanwhile, a serious burst effect (a side effect that a drug is excessively released in the initial phase of the release) has been with conventional micro/nano-particles composed of a polymer with a single phase. (Refer to Journal of Colloid and Interface Science, 2004, volume 270, pages 187-194.)

### DISCLOSURE OF INVENTION

### Technical Problem

In order to solve the above problems, one of the present inventors prepared double-layer nanoparticles with a core and shell structures to control the drug release pattern. The inventor also prepared nanoparticles which can maintain a hydrophilic environment by using a hydrophilic polymer as shell material. (Refer to the Korean Patent Application of the present inventor, Application No. 2005-59052.)

Subsequent to the above invention, the present inventors discovered that the nanoparticles for drug delivery are applicable as a carrier for a protein drug and thereby completed the present invention. Thus, the technical subject to be solved by the present invention is to make technical developments through the advantages achieved when applying the nanoparticles with core and shell structures as a carrier of a protein drug.

### Technical Solution

The present inventors has discovered that freeze-drying nanolipids comprising a mixture of lecithin and a drug in an aqueous solution of a poly(oxyethylene)-poly(oxypropylene)-poly(oxyethylene) triblock copolymer in the presence of a cryoprotectant produces novel nanoparticles with a core and shell structure, wherein the core is composed of drug-loaded lecithin nanoparticles and the shell is composed of the above type of copolymer and additives such as an emulsifier. The present inventors have also discovered that thus-obtained nanoparticles can be used as a drug carrier, and thereby completed the present invention.

The objective of the present invention is to provide a method for preparing nanoparticles with drug-loaded core and shell structures, comprising: blending nanolipids composed of lecithin extracted from soybeans as represented by formula 1, a protein drug, and a block copolymer as represented by formula 2 to form a homogeneous mixture; and freeze-drying the mixture in the presence of a cryoprotectant, wherein the nanolipids in combination with a protein drug form a core encapsulated by the triblock copolymer matrix, and wherein the polymer shell is formed on a surface of the core by an adsorption of the block copolymer. The present invention also intends to provide nanoparticles with drug-loaded core and shell structures prepared by said method.

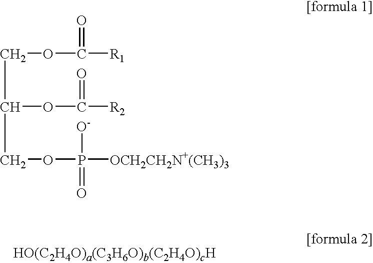

wherein b is an integer of 10 or higher, and a and c are integers such that both terminal repeat units constitute 5˜95 weight %, preferably 20˜90 weight %, of the polymer.

The more detailed description of the invention is as follows.

The nanoparticles with a lipid core and polymer shell structures for protein drug delivery of the present invention are characterized in that a nanolipid core composed of lecithin as represented by formula 1 is encapsulated by a shell composed of triblock copolymer as represented by formula 2 and that a protein drug is presented adsorbed on a surface of the lipid core.

Properties of lecithin extracted from soybeans as represented by formula 1 are known in the art and lecithin is widely used as a nutrient for patients recovering from a surgery. Because most lecithin is administered to the human body as an injection such as a Ringers solution, it does not cause any trouble in the clinical application. The studies on the delivery of drug-loaded lecithin into the human body have already been reported; however, in those studies, because drug-loaded lecithin is s single phase of lipids, a serious burst effect has been reported. (Refer to Langmuir, 2002, volume 18, pages 4061-4070.)

One example of the triblock copolymer as represented by formula 2 is a poly(oxyethylene)-poly(oxypropylene)-poly(oxyethylene) triblock copolymer, which is usually called polaxamer. Polaxamer can be prepared according to the methods disclosed in known publications, or can be available.

The polaxamer used for the present invention has a molecular weight of about 1000 to about 16000, and the properties thereof vary according to the ratio of the poly(oxyethylene) block to the poly(oxypropylene) block, that is, the ratio of a+c to b in formula 2.

Polaxamer is solid at room temperature, soluble in water and ethanol, and Polaxamers 68, 127, 237, 338 and 407 are on the market.

In the present invention, lecithin is sonicated to form nanolipids and then mixed with a protein drug to form a protein-drug-adsorbed nanolipids. Most of protein drugs have surface charge, which is positive or negative in physiological conditions depending on the isoelectric point of the protein. As can be seen above, the present invention prepares lecithin nanolipids which have negative or positive charge depending on the charges of a protein drug, and thereby forms protein-drug-adsorbed lecithin nanolipids. Thus obtained protein-drug-adsorbed lecithin nanolipids are added to a polaxamer aqueous solution wherein a cryoprotecant (e.g. trehalose) is dissolved to form a homogenous solution, and then thus-obtained solution is freeze-dried to prepare nanoparticles with protein-drug-loaded nanoparticles with core and shell structures.

In the preparation of the nanoparticles for protein drug delivery of the present invention, when a protein drug has positive charge, a method for preparing the nanoparticles for protein drug delivery comprises:
a) sonicating lecithin as represented by the formula 1 to form a nano-sized particulate lipid (lecithin nanolipid);
b) adding a cryoprotectant to an aqueous solution comprising lecithin nanolipid obtained in step a);
c) mixing the above-prepared solution comprising the lecithin nanolipid and a cryorotectant with a solution comprising triblock copolymer as represented by the formula 2 and a protein drug having positive charge; and
d) freeze-drying the solution obtained in step c).

When a protein drug has negative charge, a method for preparing the nanoparticles for protein drug delivery comprises:
a) sonicating lecithin as represented by the formula 1 to form a nano-sized particulate lipid (lecithin nanolipid);
b) mixing the lecithin nanolipid, a protein drug having negative charge and low-molecular-weight chitosan;
c) adding the mixture of the lecithin nanolipid, the protein drug having negative charges and low-molecular-weight chitosan to an aqueous solution comprising triblock copolymer as represented by the formula 2 and a cryoprotectant to form a homogenous solution; and
d) freeze-drying the solution obtained in step c).

Meanwhile, lecithin and Bovine Serum Albumin (BSA) do not form ionic interaction because both have negative surface charge. However, if the low-molecular-weight chitosan having positive charge is further added, it acts as a mediator to form ionic interaction, and thereby makes possible the formation of the BSA-adsorbed lecithin nanolipids.

Said oligo chitosan has positive charge in an acidic aqueous solution, and thus, when a mixture of oligo chitosan and 1 wt. % of acetic acid aqueous solution is added to a lecithin aqueous solution appearing negative charge, oligo chitosan is adsorbed on lecithin nanolipids by ionic interaction; here, a part of the chitosan which was not participated in ionic interaction with lecithin nanolipids enables an interaction with protein drug having negative charge. For this purpose, polyimine, polylysine, etc. can be used instead of oligo chitosan. Meanwhile, if high-molecular-weight chitosan is used, aggregation occurs and lecithin core cannot retain a nano-sized particulate form.

The ratio of lecithin to polyxamer is not particularly limited, but the ratio is generally 6:4˜1:99, preferably, 3:7˜1:9. If the ratio of lecithin/drugs/various additives: polaxamer is beyond the above range, the yield of the nanoparticles of the present invention remarkably decreases.

As an additive for adjusting particle size, biocompatible emulsifier, dispersant, surfactant, etc. can be preferably mentioned, and the type and content thereof can be suitably selected by those skilled in the art. Emulsifier, dispersant, surfactant, etc. are present at the interface of polaxamer matrix and lecithin nanoparticles.

According to the present invention, it is possible to produce, in a simple and cost-effective way, particles with core and shell structures having a desired particle size and particle size distribution and comprising various kinds of drugs and biologicals in a nanocapsule form. The particles with core and shell structures produced according to the present invention have a high stability because no organic solvent is used in its preparation process, thereby preventing any residues produced from the organic solvent.

### Advantageous Effects

According to the present invention, nanoparticles with a core and shell structure having a desired particle size, particle size distribution and protein drug loading amount can be produced in a simple and cost-effective way. The nanoparticles with a core and shell structures produced according to the present invention can be utilized as drug delivery vehicle for at least 30 types of protein drugs (i.e., regardless of whether it has positive charge or negative charge). The nanoparticles with a core and shell structures produced according to the present invention are composed of ingredients that can be used in the clinical application because no organic solvent is used in its preparation process.

### BRIEF DESCRIPTION OF THE DRAWINGS

FIG. 1 is a diagram illustrating the nanoparticles with a core and shell structures produced according to the present invention loaded with a protein drug having positive charge.

FIG. 2 is a diagram illustrating the nanoparticles with a core and shell structures produced according to the present invention loaded with a protein drug having negative charge.

FIG. 3 is a Cryo-SEM (Cryogenic-scanning electron microscopy) picture of the nanoparticles having core and shell structures produced according to the present invention.

FIG. 4 is a graph showing the release of VEGF (Vascular Endothelial Growth Factor: model protein with a positive charge) from the nanoparticles.

FIG. 5 is a graph showing the release of BSA (Bovine Serum Albumin: model protein with a negative charge) from the nanoparticles.

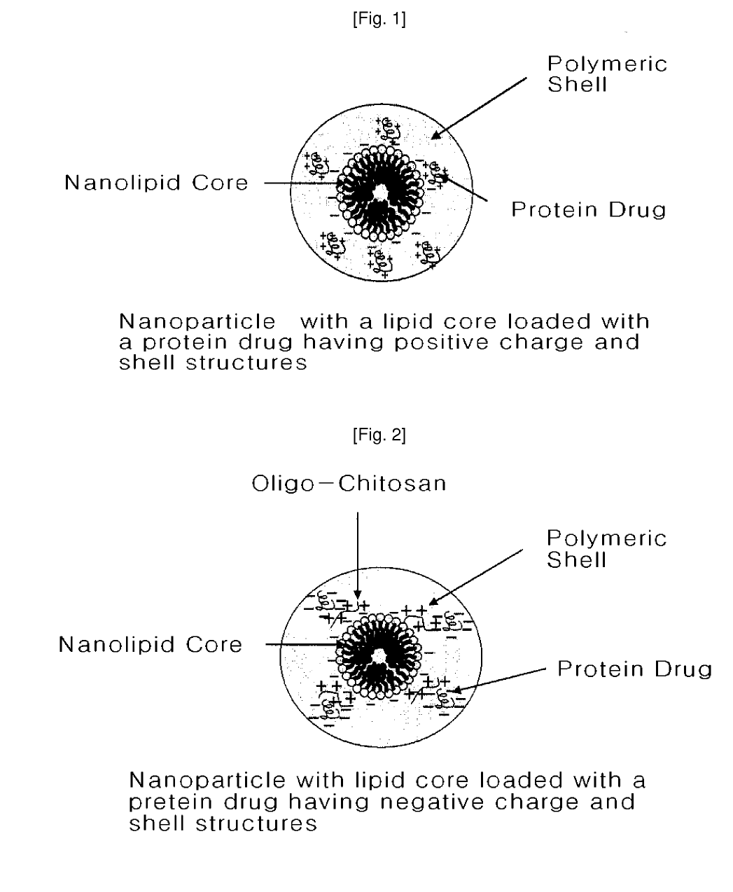

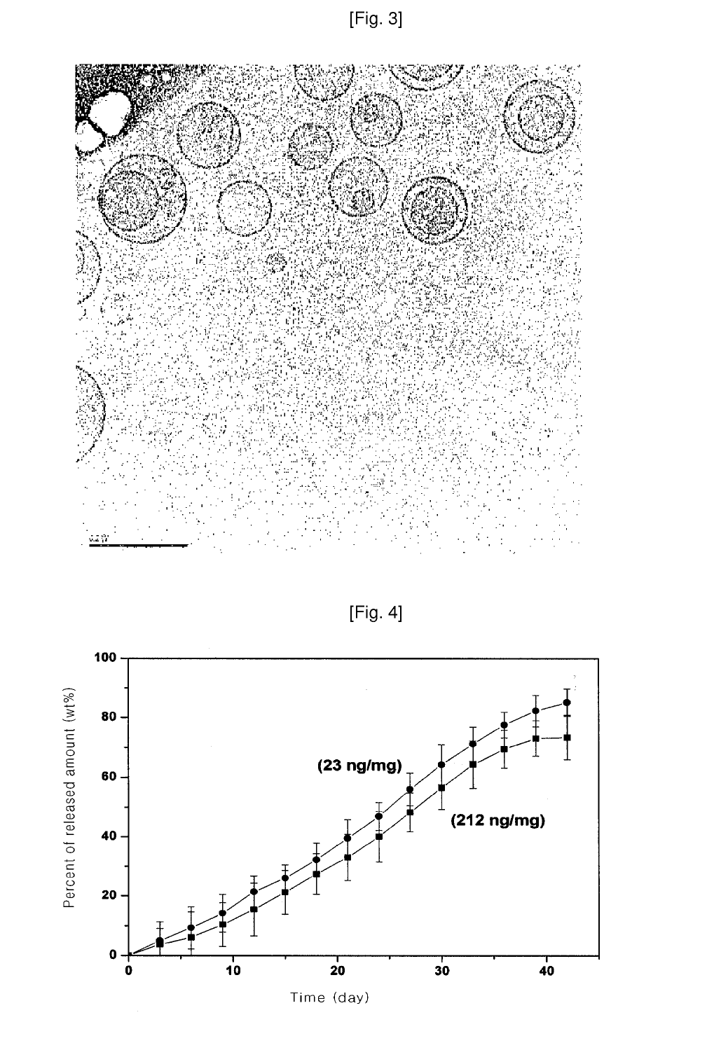

The present invention is more specifically described with reference to, but not limited to, the below examples.

### BEST MODE FOR CARRYING OUT THE INVENTION

### EXAMPLE

### Example 1

### Model Protein having Positive Charge: Vascular Endothelial Growth Factor (VEGF)

20 wt % of lecithin aqueous solution was sonicated to form lecithin nanolipids with a diameter of 65 nm. Trehalose (cryoprotectant) was added to 1 ml of the above-obtained lecithin nanolipids aqueous solution, and adjusted it at 5 wt %. On the other hand, 1 ml of 15 wt % of polaxamer (polyoxyethylene-polyoxypropylene-polyoxyethylene triblock copolymer; product name: F-127) was prepared. The above-prepared two solutions were mixed with a necessary amount of VEGF to prepare VEGF-adsorbed nanolipids in the presence of polaxamer, which is subsequently freeze-dried to provide nanoparticles with core and shell structures loaded with a protein drug having positive charge. 200 mg of nanoparticles obtained as above was added to 5 ml of phosphate buffer solution (PBS), dispersed and put in a dialysis bag, and immersed in 100 ml of PBS; then, drug release was observed at desired time. FIG. 4 shows that the sustained release of VEGF has been observed for 42 days. The released protein was analyzed by using ELISA. In the above release experiment, two types of nanoparticles were used in which 23 ng and 212 ng of VEGF were loaded, respectively, per 1 mg of nanoparticle, to observe the dependence of release pattern on loading amount and similar release pattern was observed. (See FIG. 4).

### Example 2

### Model Protein having Negative Charge: Bovine Serum Albumin (BSA) and Erythropoietin

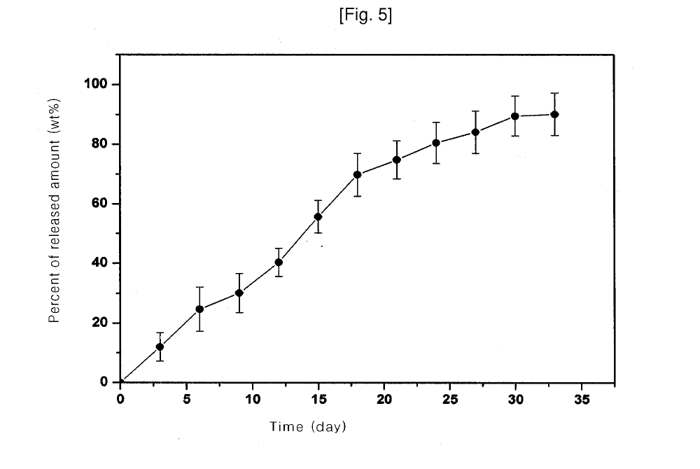

20 wt % of lecithin aqueous solution was sonicated to form lecithin nanolipids with a diameter of 65 nm. 1 mg of oligo chitosan and 1 [symbol] of BSA were added to 1 ml of the above-obtained lecithin nanolipid aqueous solution to prepare BSA-adsroded nanolipids. Then, Trehalose (cryoprotectant) was added to the thus-obtained aqueous solution, and adjusted it to 5 wt %. On the other hand, 1 ml of 15 wt % of polaxamer (polyoxyethylene-polyoxypropylene-polyoxyethylene triblock copolymer; product name: F-127) was prepared. The above-prepared two solutions were mixed to prepare BSA-adsorbed nanolipids in the presence of polaxamer, which is subsequently freeze-dried to provide nanoparticles with a core and shell structures loaed with a protein drug having a negative charge. Under the same conditions as in Example 1, the BSA release pattern was observed. FIG. 5 shows that the sustained release BSA has been observed for 33 days. The released BSA was estimated by using UV.

1. Nanoparticles with a lipid core and polymer shell structures for protein drug delivery, characterized in that
a lipid core is composed of lecithin as represented by formula 1; shell structures are composed of a triblock copolymer as represented by formula 2; said lipid core is encapsulated by said shell structure; and that a protein drug is presented adsorbed on the lipid core: 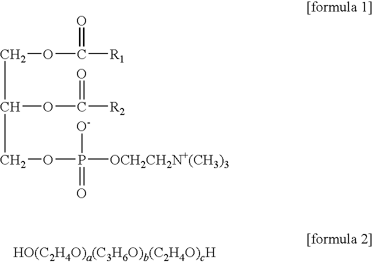
wherein, b is an integer between 10 and 150, and a and c are integers such that the sum of a and b is 80 to 350.

2. Nanoparticles for protein drug delivery according to claim 1, characterized in that the protein drug with positive charge is adsorbed on the surface of the lipid core through ionic interaction.

3. Nanoparticles for protein drug delivery according to claim 1, characterized in that the protein drug with negative charge is adsorbed on the surface of the lipid core mediated by low-molecular-weight chitosan.

4. Nanoparticles for protein drug delivery according to any of claim 1 to 3, characterized in that the ratio of lecithin to triblock copolymer is from 6:4 to 1:99.

5. Nanoparticles for protein drug delivery according to any of claim 1 to 3, further comprising additives selected from the group consisting of an emulsifier, a dispersant, a surfactant, or combinations thereof.

6. A method for preparing nanoparticles with a lipid core and polymer shell structures for protein drug delivery comprising:
a) mixing nanolipids composed of lecithin as represented by the formula 1, a protein drug and triblock copolymer as represented by the formula 2 to form a homogenous mixture; and
b) freeze-drying the mixture in the presence of a cryoprotectant.

7. A method for preparing nanoparticles with a lipid core and polymer shell structures for protein drug delivery comprising:
a) sonicating lecithin as represented by formula 1 to form nano-sized particulate lipids (nanolipids);
b) mixing the nanolipids and a protein drug;
c) adding the above-prepared mixture of the nanolipids and a protein drug to an aqueous solution comprising triblock copolymer as represented by formula the 2 and a cryoprotectant to form a homogenous solution; and
d) freeze-drying the solution obtained in step c).

8. A method for preparing nanoparticles with a lipid core and polymer shell structures for protein drug delivery, wherein a protein drug has positive charges, comprising:
a) sonicating lecithin as represented by the formula 1 to form nano-sized particulate lipids (nanolipids);
b) adding a cryoprotectant to an aqueous solution comprising the nanolipids obtained in step a);
c) mixing the above-prepared solution comprising the nanolipids and a cryorotectant with an aqueous solution comprising triblock copolymer as represented by the formula 2 and a protein drug having positive charge; and
d) freeze-drying the solution obtained in step c).

9. A method for preparing nanoparticles with a lipid core and polymer shell structures for protein drug delivery, wherein a protein drug has negative charge, comprising:
a) sonicating lecithin as represented by the formula 1 to form nano-sized particulate lipids (nanolipids);
b) mixing the nanolipids, a protein drug having negative charge and low-molecular-weight chitosan;
c) adding the mixture of nanolipids, a protein drug having negative charge and low-molecular-weight chitosan to an aqueous solution comprising triblock copolymer as represented by the formula 2 and a cryoprotectant to form a homogenous solution; and
d) freeze-drying the solution obtained in step c).

10. The method according to any of claim 6 to 9, characterized in that the ratio of lecithin to triblock copolymer is from 6:4 to 1:99.

11. The method according to any of claims 6 to 9, further comprising adding additives selected from the group consisting of an emulsifier, a dispersant, a surfactant, or combinations thereof.
